SEQUENCE LISTING (6 sequences)

SEQ ID NO: 1 - DNA, 23 bp, synthetic construct
  source 1..23: mol_type = other DNA
ttgaaagtccacctccttacaga

SEQ ID NO: 2 - DNA, 22 bp, synthetic construct
  source 1..22: mol_type = other DNA
ccggataaaaagagtacgctgg

SEQ ID NO: 3 - DNA, 21 bp, synthetic construct
  source 1..21: mol_type = other DNA
tgctgggaggtatcacccttc

SEQ ID NO: 4 - DNA, 22 bp, synthetic construct
  source 1..22: mol_type = other DNA
gctgggtttctaatctgccaat

SEQ ID NO: 5 - DNA, 19 bp, synthetic construct
  source 1..19: mol_type = other DNA
gccgacaggatgcagaagg

SEQ ID NO: 6 - DNA, 19 bp, synthetic construct
  source 1..19: mol_type = other DNA
tggaaggtggacagcgagg

(19) United States
(12) Patent Application Publication
(10) Pub. No.: US 2026/0139220 A1
(43) Pub. Date: May 21, 2026

(54) LACTICASEIBACILLUS RHAMNOSUS MBP01 AND COMBINATION THEREOF FOR PREVENTING AND TREATING IRRITABLE BOWEL SYNDROME

(71) Applicant: MAYINGLONG PHARMACEUTICAL GROUP CO.,LTD, Wuhan (CN)

(72) Inventors: Daonian ZHOU, Wuhan (CN); Wei LIU, Wuhan (CN); Meng WANG, Wuhan (CN); Hongya LIU, Wuhan (CN); Ge CHEN, Wuhan (CN); Xin MA, Wuhan (CN); Jianjian TANG, Wuhan (CN)

(21) Appl. No.: 19/283,846

(22) Filed: Jul. 29, 2025

Related U.S. Application Data
(63) Continuation of application No. PCT/CN2025/102772, filed on Jun. 23, 2025.

(30) Foreign Application Priority Data
Nov. 15, 2024 (CN) 202411631409.3

(51) Int. Cl. C12N 1/20; A61K 35/747; A61P 1/00; C12R 1/225

(52) U.S. Cl. CPC C12N 1/205; A61K 35/747; A61P 1/00; C12R 2001/225

(57) ABSTRACT

A Lacticaseibacillus rhamnosus MBP01 and a combination thereof for preventing and treating irritable bowel syndrome are provided, which relates to the technical field of microorganism. A deposit number of the Lacticaseibacillus rhamnosus MBP01 is GDMCC NO: 64725. The MBP01 can improve a weight loss trend of IBS model mice, increase food intake and feces water content, alleviate colon tissue damage, downregulate expression levels of IL-1β, TNF-α, PAR-2, and serum CORT and increase an expression level of IL-10. MBP01 with a high concentration can improve an expression level of OCC. Therefore, MBP01 has a certain positive regulatory effect on the intestinal function of IBS model mice and can effectively prevent and treat irritable bowel syndrome.

### TECHNICAL FIELD

The disclosure relates to the technical field of microorganisms, and more particularly to a Lacticaseibacillus rhamnosus MBP01 and a combination thereof for preventing and treating irritable bowel syndrome.

### STATEMENT REGARDING SEQUENCE LISTING

The sequence listing associated with this application is provided in text format in lieu of a paper copy and is hereby incorporated by reference into the specification. The name of the XML file containing the sequence listing is 25021LBZ-USP1-SL.xml. The XML file is 6394 bytes; is created on Jul. 10, 2025; and is being submitted electronically via patent center.

### BACKGROUND

Irritable bowel syndrome (IBS) is a functional intestinal disease characterized by chronic or recurrent abdominal pain and changes in bowel habits, and it lacks morphological and biochemical abnormalities. IBS is mainly characterized by a cluster of syndromes that symptoms of abdominal pain and discomfort can be alleviated or reduced after defecation, and is accompanied by changes in bowel habits and stool characteristics. The symptoms persist or recur intermittently, and have a complex pathogenesis and many influencing factors. IBS can damage intestinal mucosa, induce inflammation, destroy epithelial barrier, and cause visceral hypersensitivity.

Damage to the intestinal mucosa can lead to activation and infiltration of immune cells in the intestinal tract, thereby releasing a large number of pro-inflammatory cytokines, further aggravating inflammation in the intestinal tract, causing intestinal mucosal erosion, and in turn aggravating intestinal damage.

The inflammation is a response of the immune system to damage. The infiltration of the immune cells in the intestinal tract will lead to secretion of a large number of the pro-inflammatory cytokines, further aggravating disorder of the immune system and damaging the immune organs. Interleukin-6 (IL-6) and tumor necrosis factor-α (TNF-α) are recognized and widely studied pro-inflammatory cytokines associated with intestinal inflammation. IL-6 can promote survival of intestinal T helper 1 (TH1) and T helper 2 (TH2) cells and prevent T cell apoptosis, thereby promoting the occurrence of the intestinal inflammation. The overexpression of TNF-α can directly induce intestinal epithelial cell apoptosis and significantly weaken the intestinal barrier function. On the contrary, interleukin-10 (IL-10), as an anti-inflammatory cytokine, plays a vital role in controlling and preventing the intestinal inflammation. IL-10 is believed to block metabolism of macrophages and promote autophagy of damaged mitochondria, thereby alleviating inflammation.

The intestinal barrier function plays a vital role in the human body. Exposure of the intestinal epithelial cells to the pro-inflammatory cytokines (such as TNF-α) can lead to cell death, alter the production of secreted mucins, and damage the epithelial barrier. Occludin (OCC) is a membrane protein and a signature component of tight junction structures. Its function in the intestinal tract is usually to maintain the stability of intestinal tight junctions and ensure normal intestinal barrier function. IBS-related studies have shown that IBS with low-grade intestinal inflammation is characterized by damaged intestinal barrier function and decreased expression of OCC.

Visceral hypersensitivity is another typical clinical symptom of IBS besides low-grade inflammation. Studies have shown that visceral hypersensitivity is essentially a disorder of the peripheral and central nervous systems. In the gastrointestinal tract, mast cells are activated and release tryptase, thereby activating protease-activated-receptor-2 (PAR-2) on the surface of intestinal nerve cells, ultimately causing a sustained neural excitement state that feeds back to the gastrointestinal tract, resulting in gastrointestinal motility disorders.

Studies have shown that dietary intervention, fiber and probiotics are effective options for the treatment of IBS. Probiotics can improve intestinal microbial imbalance, regulate gastrointestinal motility, reduce visceral allergies, have a positive effect on epithelial permeability, and reduce immune mucosal activation. Lactobacillus rhamnosus GG (LGG) is a Gram-positive intestinal commensal bacterium that produces L-lactic acid, does not produce spores, and is anaerobic and acid-resistant. LGG has high acid resistance, high bile resistance, strong adhesion and good growth characteristics. LGG is one of the most widely studied probiotic strains. Its functional characteristics mainly include regulating intestinal flora, preventing and treating diarrhea, excreting toxins and enhancing the body's immunity, and has high application value.

### SUMMARY

In order to solve the above problems, the disclosure provides a Lacticaseibacillus rhamnosus, which is deposited at Guangdong Microbial Culture Collection Center (GDMCC) on Jun. 5, 2024, with a deposit number of GDMCC NO: 64725. The Lacticaseibacillus rhamnosus has shown good efficacy in preventing and/or treating irritable bowel syndrome (IBS).

On the one hand, the disclosure provides a Lacticaseibacillus rhamnosus, and a deposit number of the Lacticaseibacillus rhamnosus is GDMCC NO: 64725.

On the other hand, the disclosure provides a culture, and the culture is obtained by culturing the aforementioned Lacticaseibacillus rhamnosus.

Still on the other hand, the disclosure provides an application method of the aforementioned Lacticaseibacillus rhamnosus or culture, including:

preparing a product for preventing and treating irritable bowel syndrome by using the Lacticaseibacillus rhamnosus or the culture.

Specifically, the irritable bowel syndrome is diarrhea-predominant irritable bowel syndrome (IBS-D), constipation-predominant irritable bowel syndrome (IBS-C), unsubtyped irritable bowel syndrome (IBS-U), or mixed-type irritable bowel syndrome (IBS-M).

In an embodiment, the irritable bowel syndrome is the constipation-predominant irritable bowel syndrome.

Specifically, the product includes but is not limited to a drug, food, health food, and special purpose formula food.

In an embodiment, the food includes but is not limited to pressed candy, yogurt, cans, biscuits, chocolate, cakes, cream, cheese, cheese, milk powder, ice cream, ice cream, jam, jam, preserved fruit, preserved fruit, preserved fruit, bread, Chicken rolls, protein drink, lactobacillus beverage, vegetable protein drink, carbonated drink, coffee, and puffed food.

In an embodiment, the health food includes at least 1×10⁷ colony-forming units per gram (CFU/g) of the Lacticaseibacillus rhamnosus.

In an embodiment, the health food further includes conventional excipient for health food. The conventional excipient includes but is not limited to filler, flavoring agent, adhesive, disintegrant, lubricant, antacid, and nutritional fortifier.

In an embodiment, the health food includes lotion products, solution products, powder products and solid products.

In an embodiment, the product is a drug.

Specifically, a viable count of the Lacticaseibacillus rhamnosus in the drug is not less than 1×10⁷ CFU/g.

In an embodiment, the viable count of the Lacticaseibacillus rhamnosus in the drug can be in a range of 1×10⁷ CFU/g to 1×10¹² CFU/g.

Specifically, the drug further includes a pharmaceutically acceptable excipient.

In an embodiment, the pharmaceutically acceptable excipient is one or a combination of more than two selected from the group consisting of wetting agents, emulsifiers, preservatives, antioxidants, buffering agents, excipients, diluents, lubricants, bacteriostats, suspending agents, suspending aids, solubilizers, thickeners, stabilizers, sweeteners and spices.

In an embodiment, the pharmaceutically acceptable excipient is at least one selected from the group consisting of lactose, mannose, starch, arabic gum, calcium phosphate, alginate, gelatin, calcium silicate, polyvinylpyrrolidone, cellulose, water, syrup, methylcellulose, methyl hydroxybenzoate, propyl hydroxybenzoate, magnesium stearate, and mineral oil.

Specifically, a dosage form of the drug includes but is not limited to tablet, liquid, capsule, powder, suppository or granule.

Specifically, an administration method of the drug can be selected from oral, intravenous, local injection, intradermal injection, or subcutaneous injection.

Even still on the other hand, the disclosure provides a drug for preventing and treating irritable bowel syndrome, and the drug includes the aforementioned Lacticaseibacillus rhamnosus or the culture.

Still on the other hand, the disclosure provides a preparation method of the aforementioned drug, and the preparation method includes: culturing the Lacticaseibacillus rhamnosus to obtain the drug.

Specifically, a culture medium for culturing the Lacticaseibacillus rhamnosus includes carbon, nitrogen, and/or phosphorus sources necessary for microbial proliferation, survival, or growth. In an embodiment, the culture medium for culturing the Lacticaseibacillus rhamnosus can also include growth additives such as trace elements that are beneficial to microbial proliferation, survival or growth.

In an embodiment, those skilled in the art can use any standard or known static drying or liquid fermentation technology known in the art to prepare the “culture” or “microorganism culture” of the disclosure for use in the combination or product herein. The optimal conditions for microbial culture can depend on the specific strain. Those skilled in the art will be able to determine the appropriate nutrients and conditions. Microorganisms can be grown in aerobic, anaerobic or facultative anaerobic liquid cultures on a culture medium.

In an embodiment, the culture medium for culturing the Lacticaseibacillus rhamnosus includes but is not limited to a De Man, Rogosa and Sharpe (MRS) culture medium, or a modified MRS culture medium.

Beneficial effects of the disclosure are as follows.

(1) The MBP01 provided in the disclosure can improve a weight loss trend, and increase food intake and a feces water content in mice with the irritable bowel syndrome.

(2) The MBP01 provided in the disclosure can effectively alleviate colon damage in the mice with the irritable bowel syndrome, reduce inflammatory infiltration, and protect the structural integrity of colon tissue, thereby restoring intestinal function.

(3) The MBP01 provided in the disclosure can effectively downregulate expression levels of interleukin-1 (IL-1B), TNF-α, PAR-2, and serum corticosterone (CORT) and increase an expression level of IL-10.

(4) The MBP01 provided in the disclosure can increase an expression level of OCC and restore the intestinal barrier function damage caused by intestinal inflammation.

Preservation Description:
Name of strain: MBP01;
Deposit number: GDMCC NO: 64725;
Classification and naming: Lacticaseibacillus rhamnosus;
Deposit date: Jun. 5, 2024;
Deposit institution: Guangdong Microbial Culture Collection Center;
Abbreviation of deposit institution: GDMCC; and
Address of deposit institution: 5th floor, Building 59, No. 100 Xianlie Middle Road, Guangzhou.

### BRIEF DESCRIPTION OF DRAWINGS

FIGS. 1A-1D illustrate schematic diagrams of changes in weight and food intake in mice; where FIG. 1A is the weight of female mice; FIG. 1B is the weight of male mice; FIG. 1C is the food intake of the female mice; and FIG. 1D is the food intake of the male mice.

FIGS. 2A-2D illustrate schematic diagrams of changes in fecal water content and fecal particle count in mice; where FIG. 2A is the fecal particle count of the female mice; FIG. 2B is the fecal particle count of the male mice; FIG. 2C is the fecal water content of the female mice; and FIG. 2D is the fecal water content of the male mice.

FIGS. 3A-3B illustrate schematic diagrams of colon tissue section staining in mice; where FIG. 3A is the colon tissue of the female mice; and FIG. 3B is the colon tissue of the male mice.

FIGS. 4A-4H illustrate schematic diagrams of determination of expression levels of inflammatory cytokines in mice; where FIG. 4A is IL-10 level in the female mice; FIG. 4B is IL-10 level in the male mice; FIG. 4C is IL-1B level in the female mice; FIG. 4D is IL-1B level in the male mice; FIG. 4E is TNF-α level in the female mice; FIG. 4F is TNF-α level in the male mice;

FIG. 4G is IL-6 level in the female mice; and FIG. 4H is IL-6 level in the male mice.

FIGS. 5A-5H illustrate schematic diagrams of determination of expression levels of cytokines in mice; where FIG. 5A is OCC level in the female mice; FIG. 5B is OCC level in the male mice; FIG. 5C is PAR-2 level in the female mice; FIG. 5D is PAR-2 level in the male mice;

FIG. 5E is CORT level in the female mice; FIG. 5F is CORT level in the male mice; FIG. 5G is mast cell tryptase (MCT) level in the female mice; and FIG. 5H is MCT level in the male mice.

FIGS. 6A-6D illustrate schematic diagrams of determination of expression levels of gene in mouse colon; where FIG. 6A is PAR-2 gene level in the female mice; FIG. 6B is PAR-2 gene level in the male mice; FIG. 6C is OCC gene level in the female mice; and FIG. 6D is OCC gene level in the male mice.

### DETAILED DESCRIPTION OF EMBODIMENTS

The disclosure is further described in detail in conjunction with specific embodiments below. The following embodiments are not used to limit the disclosure, but are only used to describe the disclosure. Unless otherwise specified, experimental methods used in the following embodiments and experiment methods without specific conditions specified in the embodiments are generally conventional. Unless otherwise specified, materials, reagents and the like used in the following embodiments are commercially available.

### Embodiment 1

Mian experimental materials and reagents in the disclosure are shown in Table 1.

TABLE 1
Experimental materials and reagents

| Name | Specifications | Manufacturer | Item number |
|---|---|---|---|
| IF > 5000 Classic premix | Biochemical reagent | Yeasen Biotechnology | 11203ES08 |
| Hieff ® qPCR SYBR | | (Shanghai) Co., Ltd. | |
| Green Master Mix | | | |
| Interleukin 1 (IL-1β) | Biochemical reagent | Shanghai Enzyme-linked | 88-7013-22 |
| enzyme-linked | | Biotechnology Co., Ltd. | |
| immunosorbent assay | | | |
| (ELISA) kit | | | |
| Interleukin 6 (IL-6) | Biochemical reagent | Thermo Fisher Scientific | 88-7064-88 |
| ELISA kit | | Inc. | |
| Interleukin 10 (IL-10) | Biochemical reagent | Thermo Fisher Scientific | 88-7105-22 |
| ELISA kit | | Inc. | |
| Tumor necrosis factor-α | Biochemical reagent | Thermo Fisher Scientific | 88-7324-22 |
| (TNF-α) ELISA kit | | Inc. | |
| RNA extraction solution | Biochemical reagent | ABclonal Biotechnology | RK30129 |
| (Trizol Reagent) | | Co., Ltd. | |
| Hifair ® III 1st Strand | Biochemical reagent | Yeasen Biotechnology | 11141ES60 |
| cDNA Synthesis | | (Shanghai) Co., Ltd. | |
| SuperMix for qPCR | | | |
| Occludin (OCC) ELISA | Biochemical reagent | Shanghai Yuanju | YJ063481 |
| kit | | Biotechnology Co., Ltd. | |
| Protease-activated- | Biochemical reagent | Shanghai Yuanju | YJ037826 |
| receptor-2 (PAR-2) ELISA | | Biotechnology Co., Ltd. | |
| kit | | | |
| Monocarboxylate | Biochemical reagent | Shanghai Yuanju | YJ510082B |
| transporter (MCT) ELISA | | Biotechnology Co., Ltd. | |
| kit | | | |
| Corticosterone (CORT) | Biochemical reagent | Shanghai Yuanju | YJ037564 |
| ELISA kit | | Biotechnology Co., Ltd. | |

Main instruments and devices in the disclosure are shown in Table 2.

TABLE 2
Instruments and devices

| Name | Model | Manufacturer |
|---|---|---|
| Fully automatic enzyme-linked | VavioskanLux | Thermo Fisher Scientific Inc. |
| immunosorbent assay analyzer | | |
| Micro-spectrophotometer | Q9000M | Hangzhou Allsheng Instruments |
| | | Co., Ltd. |
| Clean bench | VS-840-2 | Suzhou Antai Airtech Co., Ltd. |
| High-pressure steam sterilization | LS-SOLD | Shanghai Shen'an Medical |
| pot | | Equipment Factory |
| Upright biological microscope | CX23 binocular | Olympus corporation of Japan |
| Electronic balance | JJ224BF | Tianjin Dat Transducer |
| | | Technology CO., LTD. |
| Ultra-low temperature freezer | DW-86L550 | Hangzhou Allsheng Instruments |
| | | Co., Ltd. |
| Pure water/ultra-pure water | UPH-1-5 | Sichuan Ulupure Ultra-Pure |
| production system | | Water Co., Ltd. |
| Table-top & High-speed | LC-LXH165A | Hangzhou Allsheng Instruments |
| refrigerated centrifuge | | Co., Ltd. |
| Table-top & High-speed | GTR16-2 | Hunan Xiangyi Laboratory |
| centrifuge | | Instrument Development Co., Ltd. |
| Dry incubator | SN-101-2QB | Hangzhou Allsheng Instruments |
| | | Co., Ltd. |

### 1.1 Experimental Method

### 1.1.1 Animal Model

25 female and 25 male C57BL/6J mice are selected and randomly divided into five groups, which are a normal group, a model group, a positive control group, a MBP01 high-dose group (MBP01-H) and a MBP01 low-dose group (MBP01-L). Each group includes 5 female mice and 5 male mice, which are respectively recorded as normal female, model female, positive female, MBP01-H female, MBP01-L female, normal male, model male, positive male, MBP01-H male, and MBP01-L male. 1-7 days after the start of the experiment, except for the normal group, all other groups are fasted for 12 hours and then enema with 0.1 milliliter (mL) 2,4,6-trinitrobenzenesulfonic acid solution (TNBS) for modeling. The MBP01-H group and the MBP01-L group are daily administered with 0.2 mL of live bacterial liquid (1×10⁹ CFU/g, 1×10⁷ CFU/g) by gavage respectively. The positive control group is daily administered with a drug linaclotide (100 micrograms per gram, which abbreviated as μg/g) by gavage after completing the modeling. An experimental period is 29 days, weight and food intake of each mouse are recorded every other day, the number of fecal particles of each mouse is recorded every two days, fresh feces of each mouse are collected and placed in a dry Eppendorf (EP) tube for weighing, the wet weight of feces is recorded, and the feces are placed in a dryer for drying and weighing to determine a fecal water content of each mouse.

$$Fecal water content ( \% ) = [ ( wet weight of feces - dry weight of feces ) / wet weight of feces ] \times 100 \% .$$

### 1.1.2 Determination of Expression Levels of Cytokines in Serum and Colon in Mice

After the end of the experimental period, whole blood is collected by taking blood from an eye socket of each mouse, and the whole blood is allowed to stand at 4 Celsius degrees (° C.) for half an hour and then centrifuged (4° C., 14000 revolutions per minute abbreviated as r/min, 15 minutes). After centrifugation, serum on an upper layer of a centrifuge tube is collected and stored in a −80° C. freezer. Colon tissue of each mouse is weighed and added into sterile saline to homogenize to obtain a homogenized solution, and the homogenized solution is centrifuged to collect a supernatant for later use. According to the specifications of the kits, ELISA kits are used to determine a content of CORT in the serum, and the expression levels of IL-1β, IL-6, IL-10, TNF-α, OCC, PAR-2 and MCT in the colon.

### 1.1.3 Colon Tissue Section Staining

The proximal colon of each mouse is cut by laparotomy about 1 centimeter (cm). Residue inside the colon is cleaned with pre-cooled physiological saline at 4° C., and the colon is cut along a longitudinal axis. The cut colon is fixed in a 4% methanol solution for 48 hours, followed by dehydrating, embedding, slicing, hematoxylin-cosin (HE) staining, and finally observing under a microscope. Images are collected and analyzed.

1.1.4 Determination of Messenger RNA (mRNA) in the Colon Tissue of Mice

The colon tissue of each mouse is weighed, cleaned with the sterile saline, and placed into a homogenization tube. The homogenization tube with the colon tissue is added with TRIzol™ for homogenization to extract RNA in the colon tissue of each mouse, to thereby obtain an RNA extraction solution. A concentration and a pureness of the RNA extraction solution are determined by using a spectrophotometer, and the RNA extraction solution is reverse transcribed into complementary DNA (cDNA) according to the specification of a reverse transcription kit. After reverse transcription, a cDNA reaction system is prepared in an 8-well strip tube, and the cDNA reaction system includes 10 microliters (μL) of reaction mixture (Mastermix), 0.4 μL of upstream primer, 0.4 μL of downstream primer, 1 μL of model cDNA and 8.2 μL of sterile nuclease-free water. After the cDNA reaction system is prepared, it is placed on a real-time quantitative polymerase chain reaction (PCR) instrument to perform amplification detection.

TABLE 3
Primer sequences

| Primer name | Primer sequence (5′-3′) |
|---|---|
| OCC | Forward primer: |
| | TTGAAAGTCCACCTCCTTACAGA |
| | (SEQ ID NO: 1) |
| | Reward primer: |
| | CCGGATAAAAAGAGTACGCTGG |
| | (SEQ ID NO: 2) |
| PAR-2 | Forward primer: |
| | TGCTGGGAGGTATCACCCTTC |
| | (SEQ ID NO: 3) |
| | Reward primer: |
| | GCTGGGTTTCTAATCTGCCAAT |
| | (SEQ ID NO: 4) |
| β-actin | Forward primer: |
| | GCCGACAGGATGCAGAAGG |
| | (SEQ ID NO: 5) |
| | Reward primer: |
| | TGGAAGGTGGACAGCGAGG |
| | (SEQ ID NO: 6) |

### 1.1.5 Data Process

The results of the experiment are expressed as mean±standard deviation (x±S), and one-way analysis of variance is used for inter group comparison. From lowercase letter a to lowercase letter e, the same lowercase letter indicates no significant difference between two groups, and the different lowercase letters indicate a significant difference between the two groups (P<0.05).

### 1.2 Results and Analysis

### 1.2.1 Results of Weight Changes in Mice

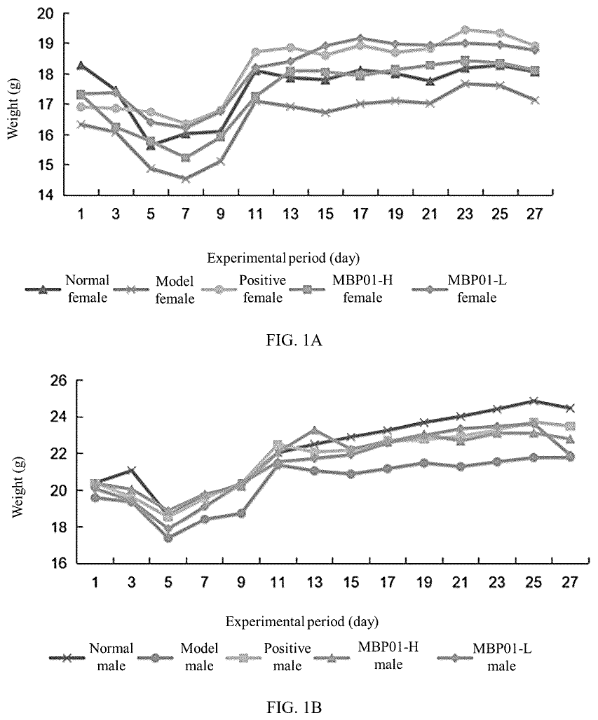

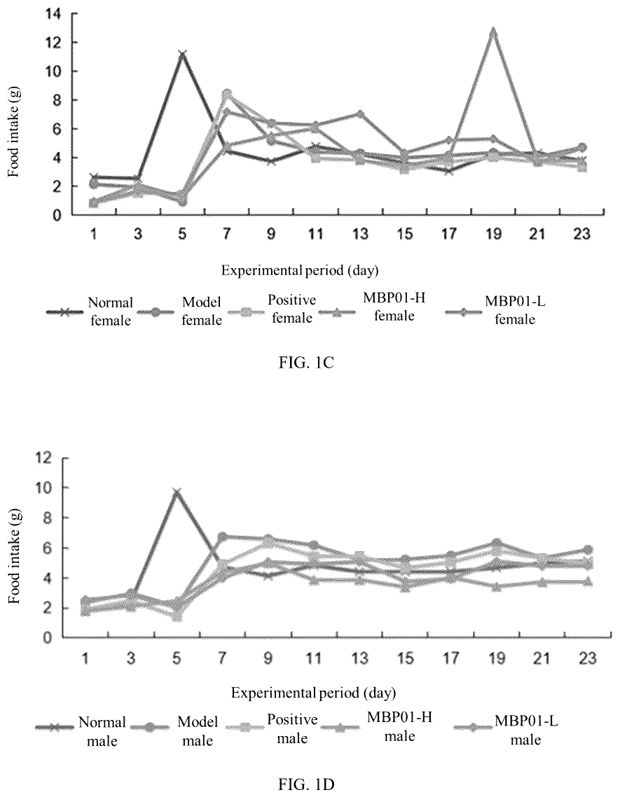

It can be seen from FIGS. 1A to 1D, compared with the normal group, the weight and food intake of mice modeled by TNBS significantly reduced. During the administration period, after drug treatment, the weight of mice in the positive control group gradually increased, with a faster growth rate. Compared with the model group, the MBP01-H group and the MBP01-L group shown significant increases in weight growth rate and food intake after intervention with the Lacticaseibacillus rhamnosus MBP01. This indicates that the irritable bowel syndrome can cause a decrease in animal diet, a decrease in animal digestive ability, and obstacles in digestion and absorption. Administration of the Lacticaseibacillus rhamnosus MBP01 can improve this symptom.

### 1.2.2 Changes in Fecal Water Content and Fecal Particle Count of Mice

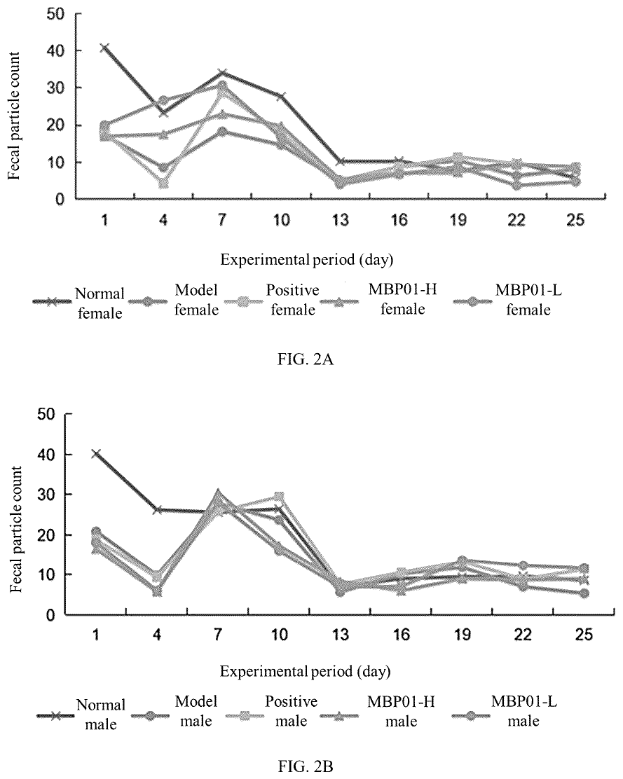

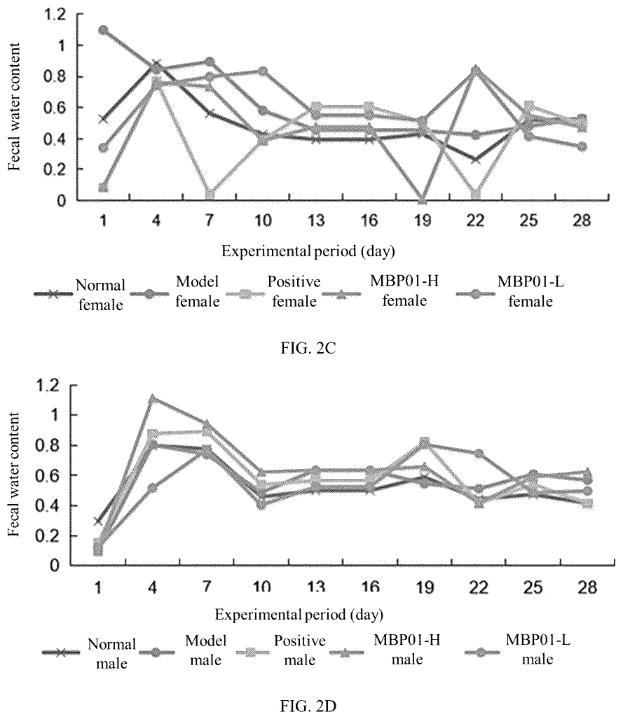

It can be seen from FIGS. 2A to 2D, after experimental modeling, except for the normal group, the fecal water content and fecal particle count of mice in other groups decreased. After intervention with drug and MBP01, the fecal water content increased and the number of fecal particles increased.

### 1.2.3 Pathological Observation of the Colon Tissue in Mice

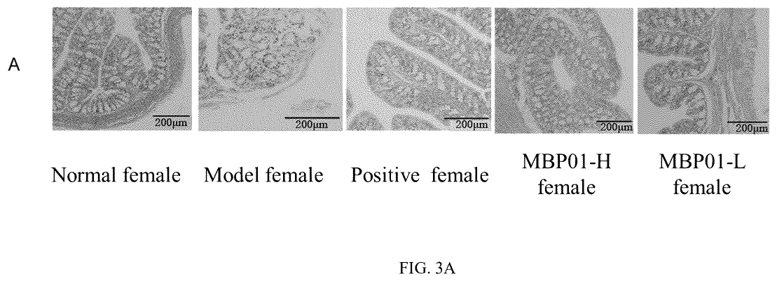

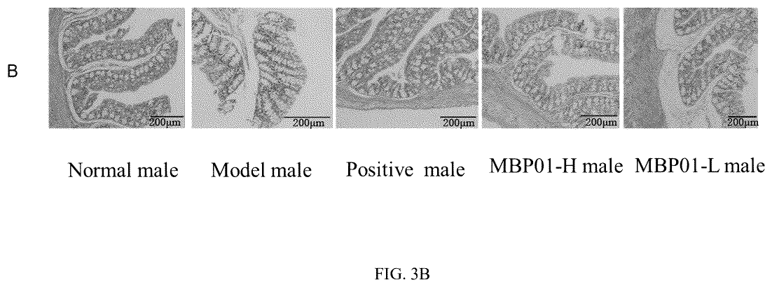

As shown in FIG. 3A and FIG. 3B, according to the results of HE pathological examination of the colon tissue, a mucosal layer, a submucosal layer, and a muscular layer of mice in the normal group have intact structures, clear textures, and no inflammatory reactions or damage to the mucosal epithelial cells, and goblet cells are arranged neatly. After modeling, inflammatory cell infiltration is observed in the colon of mice, and the submucosal structure is loose with some edema, and the arrangement of the goblet cells is loose. Compared with the model group, the positive control group, the MBP01-H group and the MBP01-L group shown reduced colon damage and inflammation infiltration in mice, indicating that treatment with MBP01 can alleviate colon damage caused by IBS to some extent.

### 1.2.4 Determination of Expression Levels of Inflammatory Cytokines

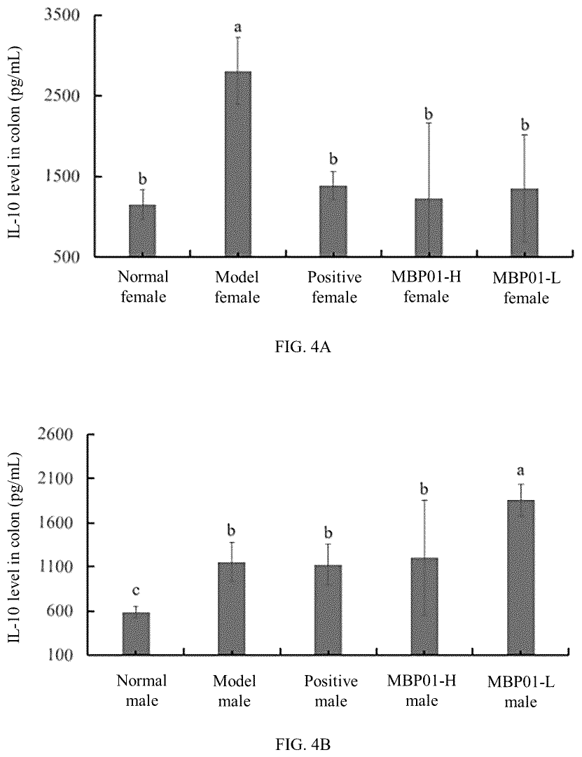

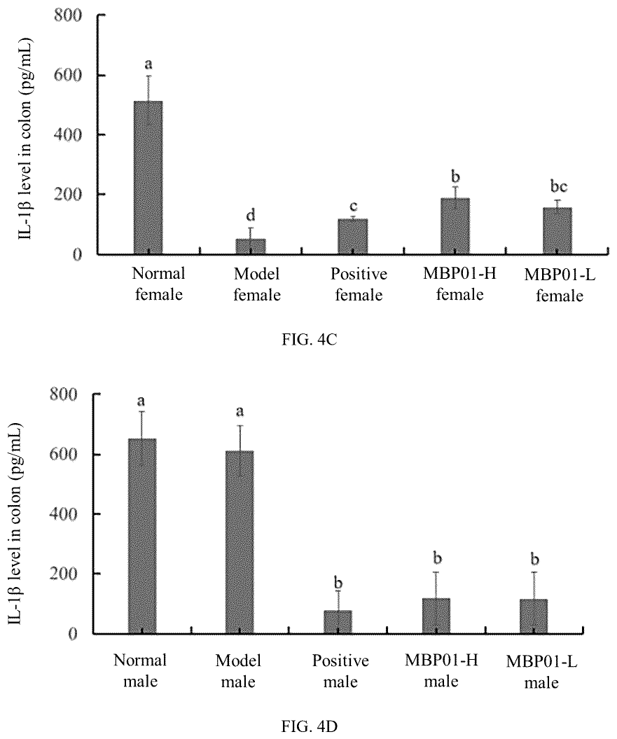

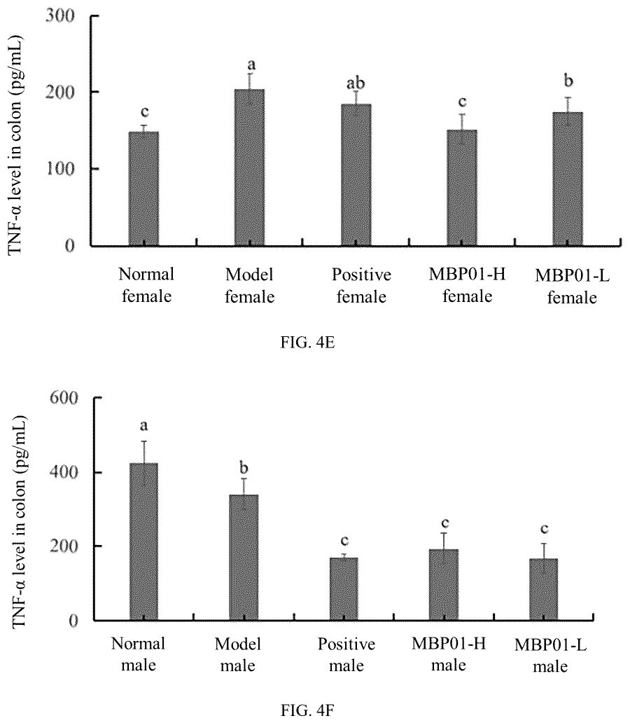

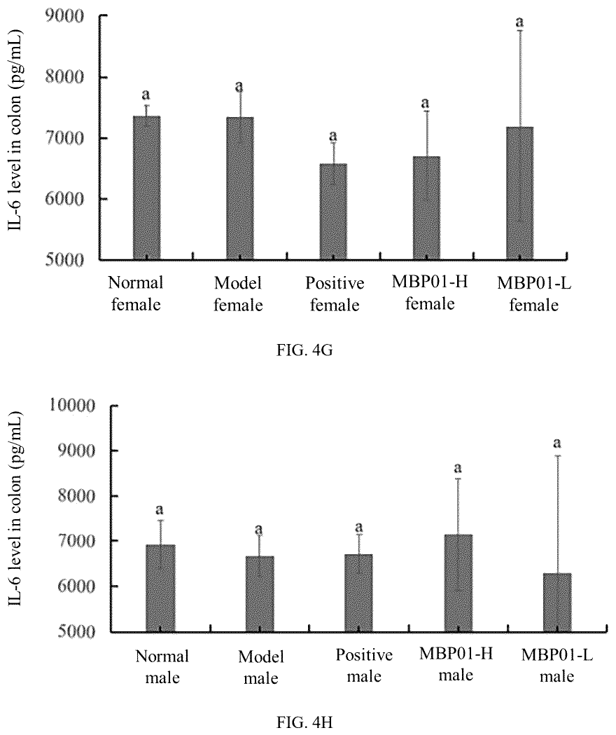

As shown in FIGS. 4A to 4H, compared with the normal group, the expression levels of IL-10 in female mice and in male mice, and TNF-α in female mice in the model group significantly increased (P<0.05), the expression levels of IL-1B in female mice and TNF-α in male mice in the model group decreased (P<0.05), and the expression level of IL-1B in male mice in the model group does not change. Compared with the model group, the expression levels of IL-1B in in female mice and in male mice, TNF-α in female mice and male mice, and IL-10 in female mice in the MBP01-H group and the MBP01-L group significantly decreased (P<0.05), and the expression level of IL-10 in male mice in the MBP01-L group significantly increased (P<0.05). The IL-6 did not show a significant trend in this experimental study and did not have statistical significance (P>0.05).

### 1.2.5 Determination of Expression Levels of MCT, PAR-2 and OCC in Colon, and CORT In Serum of Mice

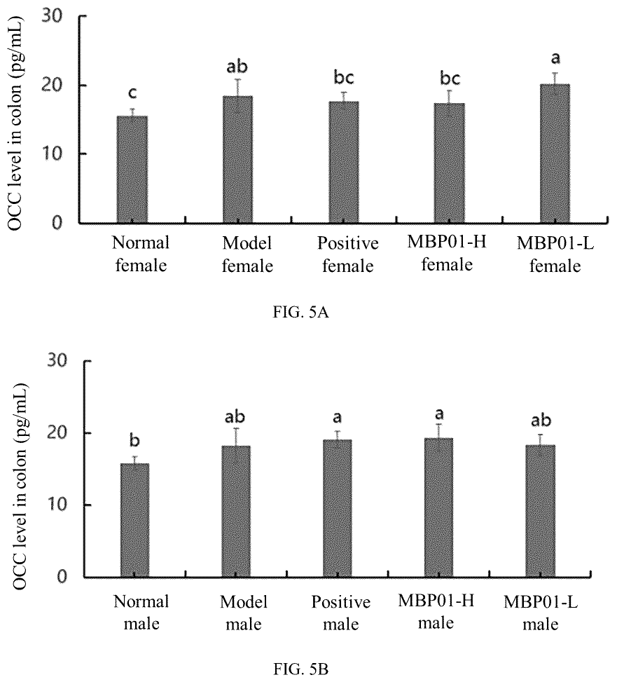

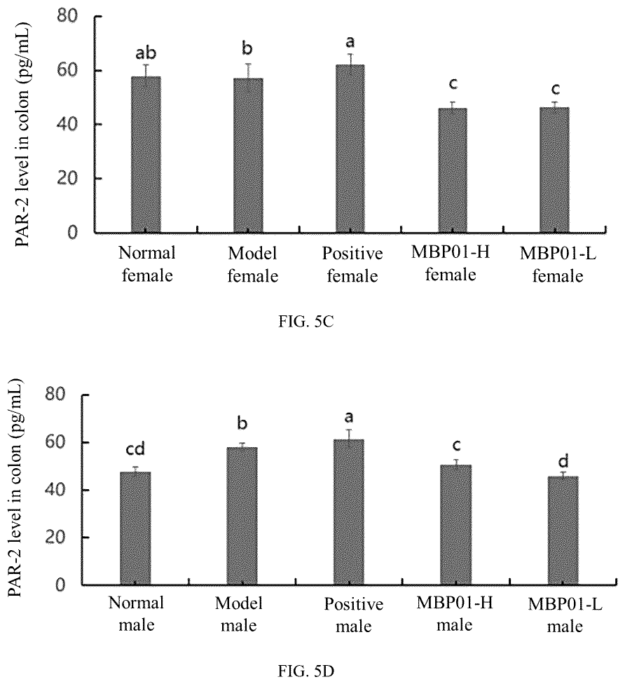

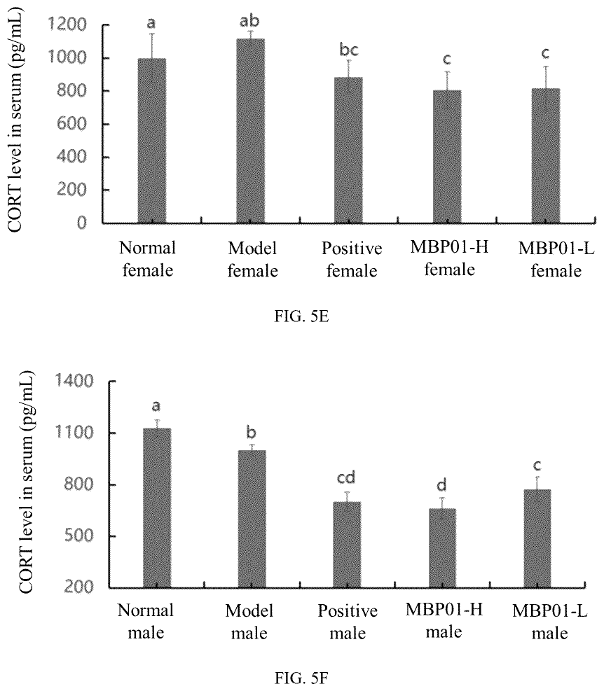

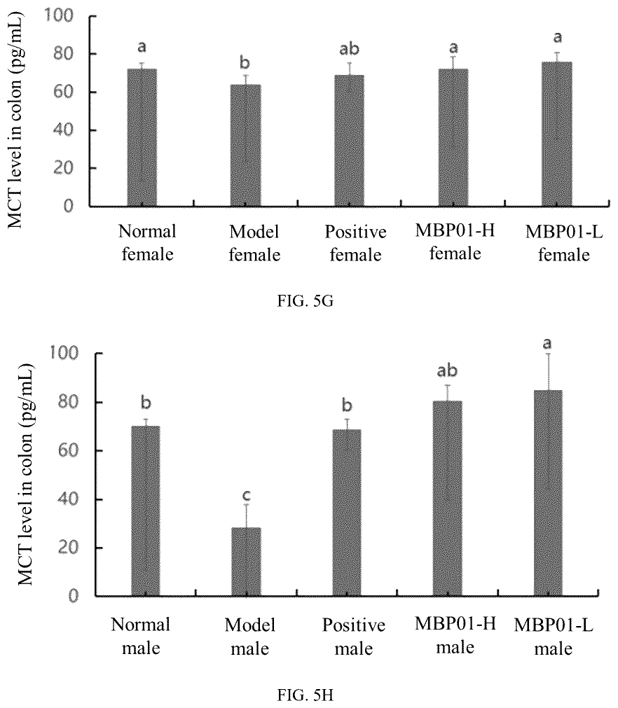

As shown in FIGS. 5A to 5H, compared with the normal group, the expression levels of OCC and PAR-2 in female mice and in male mice, and CORT in female mice in the model group significantly increased (P<0.05), and the expression levels of MCT in female mice and in male mice, and CORT in male mice in the model group significantly decreased (P<0.05). Compared with the model group, the expression levels of PAR-2 and CORT in the MBP01-H group and the MBP01-L group significantly decreased (P<0.05), the expression level of MCT in the MBP01-H group and the MBP01-L group significantly increased (P<0.05), and the expression level of OCC shown no significant trend of change.

### 1.2.6 Determination of Expression Levels of PAR-2 and OCC in Colon of Mice

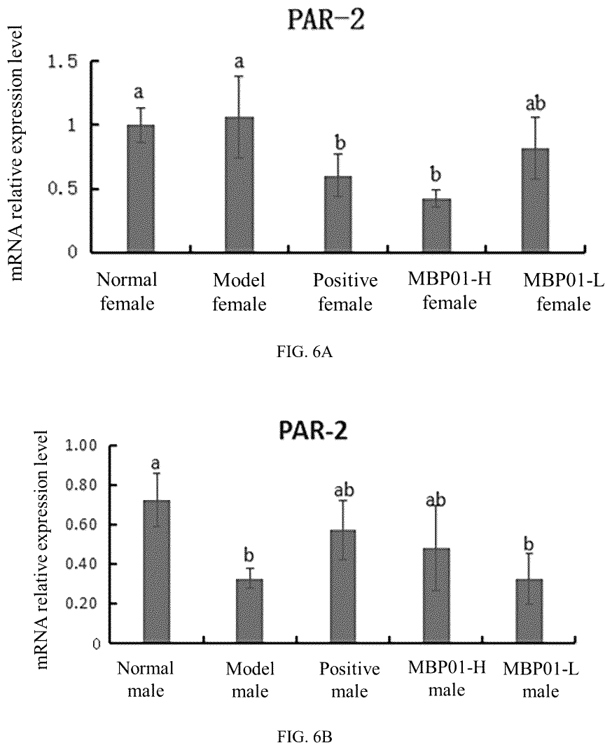

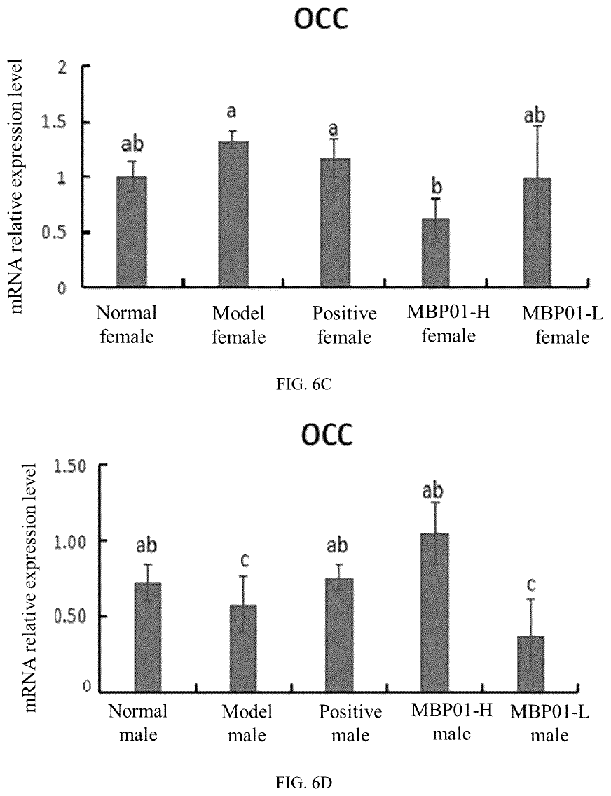

As shown in FIGS. 6A to 6D, the analysis of the gene expression levels of OCC and PAR-2 in the colon of mice shown that compared with the normal group, the gene expression levels of OCC and PAR-2 in male mice in the model group significantly decreased (P<0.05). Compared with the model group, MBP01-H can significantly reduce the gene expression levels of OCC and PAR-2 in the female mice (P<0.05), increase the gene expression levels of OCC and PAR-2 in the male mice; while no significant trend is observed in the gene expression levels of OCC and PAR-2 in the MBP01-L group.

### 1.3 Analysis and Discussion

In the disclosure, it can be found that the state of mice in the model group has undergone significant changes compared to the mice in the normal group. The mice in the model group shown a manic state in the early stage of modeling, but in the later stage, they transformed into a slow response state, accompanied by weight loss, reduced diet, decreased number of fecal particles, and decreased fecal water content. When drugs and MBPOI are used for intervention, the overall physical signs of the mice are significantly improved, the activity levels of the mice increased, their food intake significantly increased, and the weight of the mice increased. The number of fecal particles increased, and the fecal water content increased. This indicates that the Lacticaseibacillus rhamnosus MBP01 can improve the condition of mice with IBS and has a certain therapeutic effect.

In the colon of IBS model mice, a large number of inflammatory cell infiltration can be observed in the colon tissue, with loosely arranged goblet cells and loosely connected intercellular connections. After MBP01 intervention, inflammatory infiltration in colon tissue of mice is reduced, intercellular connections are tight, and the goblet cells are arranged neatly. This indicates that the Lacticaseibacillus rhamnosus MBPOI can effectively alleviate colon damage in mice with IBS, reduce inflammatory infiltration, protect the structural integrity of colon tissue, and restore intestinal function.

In order to further investigate the alleviating effect of MBP01 on IBS with low-grade inflammation, the expression levels of pro-inflammatory cytokines and anti-inflammatory cytokines in the colon of mice are studied. The results shown that TNBS enema will induce autoimmune disorders in mice, and the experiment found that the expression level of TNF-α in the colon of female mice significantly increased. This indicates that the intestinal immune homeostasis of mice is affected, and their intestinal tracts exhibit inflammatory symptoms. However, after treatment with the Lacticaseibacillus rhamnosus MBP01, the expression levels of TNF-α and IL-1B in the colon of mice significantly decreased, while the expression level of the IL-10 in male mice in the MBP01-L group significantly increased. This indicates that the Lacticaseibacillus rhamnosus MBP01 has the effect of alleviating IBS with low-grade intestinal inflammation symptoms.

In the disclosure, the expression level of OCC in colon of mice is determined by PCR, and the results shown that compared with the normal group, and the expression level of OCC in male mice in the model group significantly decreased. After oral administration of the Lacticaseibacillus rhamnosus MBP01, the expression level of OCC in male mice in the MBP01-H group significantly increased, demonstrating that Lacticaseibacillus rhamnosus MBP01 with high-dose can restore intestinal barrier function damage caused by intestinal inflammation.

In the disclosure, it is found that compared with the normal group, the expression levels of PAR-2 in female mice and in male mice, and CORT in female mice in the model group significantly increased. After treatment with MBP01, the expression levels of PAR-2 and CORT in the MBP01-H group and the MBP01-L group significantly decreased, indicating that Lacticaseibacillus rhamnosus MBP01 may have the effect of improving visceral allergy symptoms in mice.

### Comparative Embodiment 1

The strain studied for its effect on IBS in the comparative embodiment 1 is Lacticaseibacillus rhamnosus 4F225 with a deposit number China General Microbiological Culture Collection Center (CGMCC) NO: 26437. The modeling process and dosage (1×10⁹ CFU/kg) for IBS are the same as in the embodiment 1, with the only difference being the specific strain.

Experimental results: compared with the model group, there is no significant change in the expression level of TNF-α in female mice after intervention with Lacticaseibacillus rhamnosus with the deposit number CGMCC NO: 26437.

What is claimed is:

1. A Lacticaseibacillus rhamnosus, wherein a deposit number of the Lacticaseibacillus rhamnosus is Guangdong Microbial Culture Collection Center (GDMCC) NO: 64725.

2. A culture, wherein the culture is obtained by culturing the Lacticaseibacillus rhamnosus as claimed in claim 1.

3. An application of the Lacticaseibacillus rhamnosus as claimed in claim 1, comprising:
preparing a drug for preventing and treating irritable bowel syndrome by using the Lacticaseibacillus rhamnosus.

4. An application of the culture as claimed in claim 2, comprising:
preparing a drug for preventing and treating irritable bowel syndrome by using the culture.

5. The application as claimed in claim 3, wherein the irritable bowel syndrome is diarrhea-predominant irritable bowel syndrome (IBS-D), constipation-predominant irritable bowel syndrome (IBS-C), unsubtyped irritable bowel syndrome (IBS-U), or mixed-type irritable bowel syndrome (IBS-M).

6. The application as claimed in claim 3, wherein a viable count of the Lacticaseibacillus rhamnosus in the drug is not less than 1×10⁷ colony-forming units per gram (CFU/g).

7. The application as claimed in claim 3, wherein the drug further comprises a pharmaceutically acceptable excipient.

8. The application as claimed in claim 3, wherein a dosage form of the drug is tablet, liquid, capsule, powder, suppository or granule.

9. A drug for preventing and treating irritable bowel syndrome, wherein the drug comprises the Lacticaseibacillus rhamnosus as claimed in claim 1.

10. A drug for preventing and treating irritable bowel syndrome, wherein the drug comprises the culture as claimed in claim 2.

11. A preparation method of the drug as claimed in claim 9, comprising:
culturing the Lacticaseibacillus rhamnosus to obtain the drug.
